(19) United States
(12) Patent Application Publication
(10) Pub. No.: US 2024/0374469 A1
(43) Pub. Date: Nov. 14, 2024

(54) SYSTEMS AND METHODS RELATED TO MANUALLY SUPPORTED STIMULATORS

(71) Applicant: JOANNA FRACZEK, MILWAUKEE, WI (US)

(72) Inventor: JOANNA FRACZEK, MILWAUKEE, WI (US)

(21) Appl. No.: 18/688,923

(22) PCT Filed: Sep. 1, 2022
(86) PCT No.: PCT/US22/75810

§ 371 Date: Mar. 4, 2024

Related U.S. Application Data
(60) Provisional application No. 63/344,826, filed on May 23, 2022.
(60) Provisional application No. 63/240,488, filed on Sep. 3, 2021.

(51) Int. Cl. A61H 23/02

(52) U.S. Cl. CPC A61H 23/02; A61H 2201/0153; A61H 2201/0157; A61H 2201/0207; A61H 2201/0214; A61H 2201/10; A61H 2201/1207; A61H 2201/1638; A61H 2201/165; A61H 2201/169; A61H 2201/5007

(57) ABSTRACT

Systems and methods according to the present invention provide and utilize finger extensions for bodily stimulation. An extension may be utilized on a single finger of a human hand, or multiple extensions may be used, each on one finger of the hand. Each extension preferably includes at least one stimulation feature, such as altered surface texture, added surface texture, vibration, and/or a heating element. Electrically powered stimulation features are powered by a rechargeable power source, and may be controlled by a microprocessor, which may be wirelessly remote controlled.

### RELATED APPLICATIONS

This application is a national phase application of international patent application PCT/US22/75810, filed Sep. 1, 2022, entitled “Systems and Methods Related to Manually Supported Stimulators,” which claims the benefit of United Stated Provisional Patent Applications Ser. No. 63/240,488, filed Sep. 3, 2021, and Ser. No. 63/344,826, filed May 23, 2022, both titled “Systems and Methods Related to Manually Supported Stimulators,” all of which are incorporated herein by reference in their entirety.

### BACKGROUND OF THE INVENTION

This invention relates generally to a manually supported (e.g., coupled to a wrist and/or hand) stimulator for use in bodily stimulation for relaxation, therapy, and/or sexual stimulation. Generally, known stimulators make use of a primary type of stimulation, such as vibration, temperature change, electric impulse, or mechanical texture (e.g., ribs, bumps, etc.). Each type of stimulation can yield unique advantages in areas such as muscle relaxation, recovery and repair, chronic pain and fatigue treatment, stress reduction, improved circulation, and sexual satisfaction.

Many stimulation systems and devices have been developed with the last advantage in particular in mind. These include both standalone devices and devices that are meant to pair with a specific part of the human body. For instance, numerous disclosures have been made for systems involving gloves and stimulation. However, most, if not all, such systems involve only one form of stimulation. These systems are also fairly small in size, only able to stimulate a small area of the body (e.g., no larger than the size of the human hand wearing the device) at any given time. Therefore, the field is in need of an improved stimulation system that can provide multiple forms of stimulation and may simultaneously stimulate many, and expansive, areas of a human body at once.

### SUMMARY OF THE INVENTION

Embodiments of systems and methods according to the present invention relate generally to manually supported stimulation devices. More specifically, devices (some preferably selectively structurally and/or operationally configurable) for providing stimulation to various areas of the body through touch, vibration, electric pulse, temperature change, and/or texture variations.

An embodiment of the system for manually supported stimulation according to the present invention has at least one elongated member, which includes a finger sheath configured to receive a human finger and be at least partially supported by the human finger, a body extending from the finger sheath to a free end, the finger sheath and body extending generally coaxially along a longitudinal length, the longitudinal length being at least three inches, and a first stimulation feature supported by the body.

According to an aspect of a system for manually supported stimulation according to the present invention, the longitudinal length is three inches to twelve inches.

According to another aspect of a system for manually supported stimulation according to the present invention, the longitudinal length is five inches to twelve inches.

According to still another aspect of a system for manually supported stimulation according to the present invention, the first stimulation feature is located closer to the free end than to the finger sheath.

According to yet another aspect of a system for manually supported stimulation according to the present invention, a power supply is electrically coupled to the first stimulation feature.

According to a further aspect of a system for manually supported stimulation according to the present invention, the system further includes a strap and the power supply is supported by the strap.

According to a still further aspect of a system for manually supported stimulation according to the present invention, the strap is configured to be worn on one or more of a human wrist and a human hand.

According to a still further aspect of a system for manually supported stimulation according to the present invention, the system further includes a connector band in mechanical and electronic communication with the strap and the elongated member.

According to a still further aspect of a system for manually supported stimulation according to the present invention, the power supply includes a rechargeable battery.

According to a still further aspect of a system for manually supported stimulation according to the present invention, the first stimulation feature and the power supply are electrically coupled by a wire that extends along a majority of the longitudinal length, the wire further capable of supporting the body in a selective plastically deformed position.

According to a still further aspect of a system for manually supported stimulation according to the present invention, the first stimulation feature includes at least one of a vibration motor, a heat pad, an electrically conductive surface, and a textured area.

According to a still further aspect of a system for manually supported stimulation according to the present invention, the elongated member includes a second stimulation feature.

According to a still further aspect of a system for manually supported stimulation according to the present invention, the second stimulation feature includes at least one of a vibration motor, a heat pad, an electrically conductive surface, and a textured area.

According to a still further aspect of a system for manually supported stimulation according to the present invention, the second stimulation feature is different than the first stimulation feature.

According to another embodiment of a system for manually supported stimulation according to the present invention, it includes a finger sheath configured to receive a human finger and be at least partially supported by the human finger, a body extending from the finger sheath to a free end, the finger sheath and body extending generally curvilinearly coaxially along a longitudinal length, the longitudinal length being at least three inches, and a first stimulation feature supported by the body. The body includes a first extension member coupled to the finger sheath and a second extension member coupled to the first extension member and forming the free end.

According to an aspect of an embodiment of a system for manually supported stimulation according to the present invention, it includes one or more additional extension members disposed between the first extension member and the second extension member.

According to another aspect of an embodiment of a system for manually supported stimulation according to the present invention, the first and second extension members are mechanically coupled.

According to still another aspect of an embodiment of a system for manually supported stimulation according to the present invention, the first and second extension members are electrically coupled.

According to yet another aspect of an embodiment of a system for manually supported stimulation according to the present invention, at least one of the additional extension members is electrically coupled to the first extension member.

According to a further aspect of an embodiment of a system for manually supported stimulation according to the present invention, the first stimulation feature is an active stimulation feature.

According to a still further aspect of an embodiment of a system for manually supported stimulation according to the present invention, the first stimulation feature is at least one of a vibrating motor, a heating pad, an electrically conductive surface, and a light.

According to a still further aspect of an embodiment of a system for manually supported stimulation according to the present invention, the first stimulation feature is a passive stimulation feature.

According to a still further aspect of an embodiment of a system for manually supported stimulation according to the present invention, the first stimulation feature is at least one of a textured area and an ice cube.

### BRIEF DESCRIPTION OF THE DRAWINGS

FIG. 1 is a top plan view of a first embodiment of a stimulation system according to the present invention on a user's right hand.

FIG. 2 presents three optional stimulator end portions according to the present invention, to be substituted within dotted line 2 of FIG. 1.

FIG. 3 is a bottom plan view of the embodiment of FIG. 1, on a user's left hand.

FIG. 4 is a right side elevation view of an embodiment of an elongated member according to the present invention.

FIG. 5 provides optional cross-section views taken along line 5-5 of FIG. 4, with cross-hatching removed so as to show detail.

FIG. 6 is a representative cross-section elevation view taken along line 6-6 of FIG. 1.

FIG. 7 is a top plan view of a second embodiment of a stimulation system according to the present invention.

FIG. 8 is an exploded view of a third embodiment of a stimulation system according to the present invention.

FIG. 9 is a cross-sectional view taken along the 9-9 line of FIG. 8.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

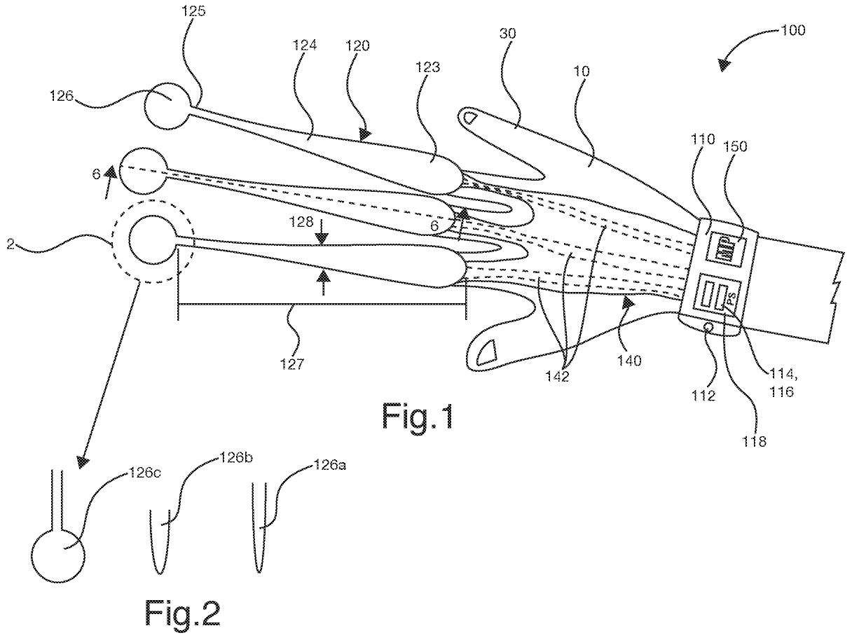

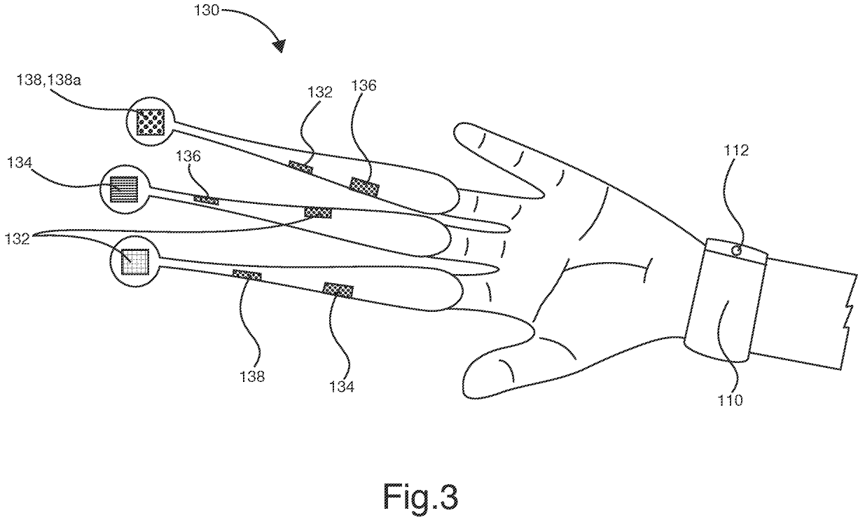

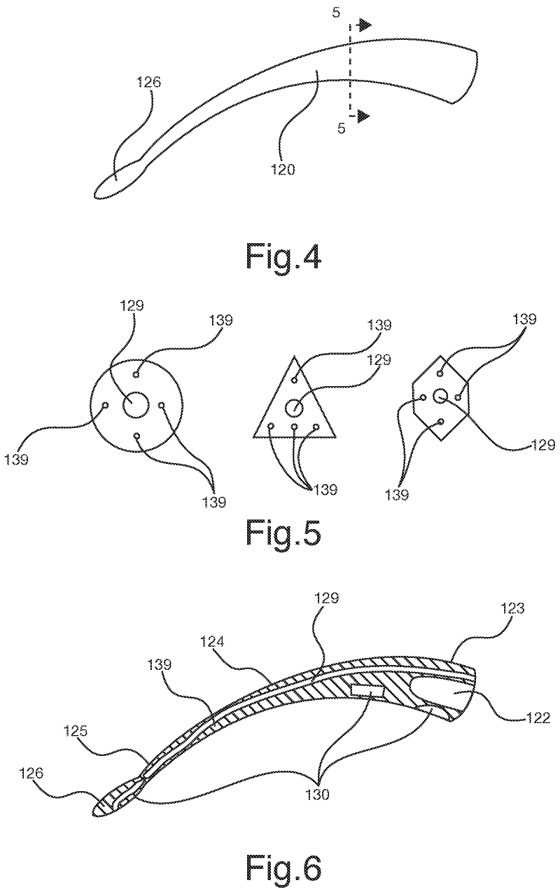

Turning now to Figures, systems and methods related to manually supported stimulation systems according to the present invention can be seen. In FIG. 1, a first embodiment for a body stimulation system 100 generally comprises a mounting strap 110 in electric communication with at least one elongated member 120 through a connector band 140. The system 100 provides various means for stimulating through vibration, temperature change, electric impulse, and texture. The system 100 may optionally further comprise a microprocessor 150 and related smart device application (not shown) in wireless communication with the microprocessor 150 (such as through a Bluetooth® connection) for distanced control. Generally, the system 100 engages the use of a user's hand 10 to allow the user control over the area and intensity of stimulation as desired. The system 100 does not depend on which hand 10 is used, thus the system 100 may be used in conjunction with either the user's right hand or left hand, or a system could be provided for each hand.

The strap 110 of the system 100 is preferably configured to fit around a user's wrist 20 to anchor the system 100 to the user. Thus, the strap 110 is preferably a unitary piece of material which may fit comfortably on the wrist 20. For example, the strap 110 may include a fastener 112 (e.g. hook and loop, button, hook, etc.) to prevent the strap 110 from detaching from the user's wrist 20. The fastener 112 may also optionally allow the user to adjust the tightness of the strap 110 against the wrist 20. Other embodiments of the strap 110 are also contemplated, including embodiments lacking a fastener (e.g. an elastic wristband) or embodiments with multiple fasteners. The strap 110 preferably comprises a durable material (e.g. leather, silicone, cotton, cloth, etc.) that is comfortable for a user to wear.

Preferably affixed to the strap 110 is a power source 114. The power source 114 ideally is capable of supplying direct current power. The power source 114 preferably comprises a battery 116. The battery 116 is preferably a conventional battery known in the art (e.g., rechargeable lithium-ion battery). The battery 116 is also preferably rechargeable, although non-rechargeable batteries are also contemplated. The power source 114 may be permanently affixed to the strap 110 through the use of an adhesive or may be removably affixed via a mount 118 (not shown). In embodiments where the power source 114 is rechargeable, an electronic port 119 may optionally be included to connect the power source 114 to an outside power source via cable for charging, or it may be wirelessly (e.g., inductively) rechargeable.

The one or more elongated members 120 are configured to be supported by the user's fingers 30. One elongated member 120 preferably is mateable with one finger 30. Although the systems shown feature three elongated members 120, the system 100 may feature anywhere between 1 and 5 elongated members 120. Each elongated member 120 comprises a finger sheath 122, a member body 124, and one or more stimulation features 130. The finger sheath 122 preferably surrounds a pocket in the member body 124 extending into the member body 124 from a first proximal end 123. In use, the sheath 122 preferably receives a finger 30 of a user, allowing the user to direct the elongated member 120 to a desired location through movement of the finger. Ideally, the member body 124 includes a unitary piece of durable material that allows for protection from wear during use, maintenance, and storage, while also allowing for comfort of the user (e.g., electrically conductive or nonconductive silicone rubber).

Ideally, the member body 124 is shaped similarly to a human finger (e.g., to act as a finger extension), with a width 128 that tapers from the first proximal end 123 to a second distal end 125. The first end 123 is preferably only a little wider than the user's finger 30, so as to allow a snug fit in the finger sheath 122. However, while the body 124 may have a cross-section of any geometric or irregular shape, as exemplary shown in FIG. 5, the elongated member 120 preferably has a circular cross-section. Further, the member body 124 preferably comprises a length 127 between the first end 123 and a second end 125 that is much longer than (1.5 to 3 times the length of) a human finger on which it is worn. Exemplary lengths 127 range preferably from about three inches to about twelve inches, and more preferably about five inches to about twelve inches. This extended length 127 allows a user to reach areas of their own human body or another human body with the elongated members 120 that would otherwise be unreachable and/or to effectively increase the span of reach between distal ends. That is, a span of a male human hand (i.e., measured from tip of outstretched pinky finger to tip of outstretched thumb) generally is thought to average about 9.5 inches. Accordingly, disposing a body 124 on a pinky finger and a body 124 on a thumb of the same hand could increase the span from about 50% to about 150%, with an increase of about 60% to about 100% being preferred, and about 75% being most preferred. Thereby, an average male hand span could be increased from about 9.5 inches to from about 14.25 inches to about 23.75 inches, which would allow for a span of a single human hand, with one or more member bodies 124 extending from finger(s), to cover an entire width of an average human torso. Accordingly, a system according to the present invention preferably includes elongated members 120 that significantly increase the effective hand size of the user, allowing the user to affect a significantly larger area of a human body with only a single hand.

The member body 124 may be provided as a solid unitary member (e.g., solid rubber cross-section), with a stimulating feature (e.g., fabrics or directional fibers, as further discussed below) coupled at or near the distal end, or even formed as part of and integrally with the distal end (e.g., bumps or ridges). Alternatively, the member body 124 may include a solid core wire 129 made of a bendable, yet electrically conductive material (e.g. 12- or 14-gauge copper). The solid core wire 129 preferably runs through a majority of the length 127 of the body 124, allowing the user to plastically deform the body 124 into a desired shape, the body 124 thereby substantially retaining that shape. The solid core wire 129 may further serve an electrical purpose, as more fully describe below.

Located proximate to the second (distal) end 125, a body tip portion 126 terminates the body 124 of the elongated member 120. As seen in FIG. 2, the tip portion 126 may be provided in various forms, including a pointed tip 126a, rounded tip 126b, or a pad tip 126c. The pad tip 126c is preferably a flat pad which includes a surface area in or on which to place one or more stimulation features 130. The other tip configurations may include one or more stimulation features 130 as well. One embodiment of the system 100 may include multiple (two to five) elongated members 120, each with the same type of body tip 126, and same stimulation features 130. Additionally or alternatively, one or more members 120 may include a different body tip 126, different cross-section, or different stimulation features 130 (or different orientation thereof) than one or more of the other members 120. Ideally, in use, the body tip 126 is placed in contact with a human body (i.e., the wearer's body or a different body) where stimulation is desired, although other portions of the member body 124 that contain stimulators 130 may also or alternatively be placed in contact with the body.

The one or more stimulation features 130 may be located anywhere on the outer surface (or at least perceptible through or on the outer surface) of the body 124 and placed in contact with an animal (e.g., human) body to provide stimulation. These stimulation features 130 may include one or more vibration motors 132, one or more heat pads 134, one or more electrodes 136, and/or one or more textured areas 138. A vibration motor 132 may be positioned at any locations along the length 127, preferably embedded in the body 124) and positioning will affect the vibration intensity provided at the tip 126. For more intense vibration stimulation at the body tip 126, motors 132 may be placed proximate to the second end 125 of the member body 124, while motors 132 located further from the distal end 125 will generally cause less intense vibrations at the body tip 126. The vibration motors 132 may be conventional vibration motors known in the art of any shape, but are preferably a button-type vibration motor (e.g. a BestTong 1.5V DC Button vibration motor) or an encapsulated vibrating motor. The one or more vibration motors 132 are preferably in electric communication with the power source 114, such as through wires 139 running preferably through the member body 124 and/or by connection to the conductive solid core wire 129.

As temperature changes can be both soothing and stimulating, the member body 124 may optionally feature one or more heat pads 134 located at various positions along the outer surface of the body 124, though preferably located at the body tip 126, if included. Additionally or alternatively, a heat pad may be mounted on or embedded into the finger sheath 122. Like the vibration motors 132, the heat pads 134 are preferably in electric communication with the power source 114, such as through wires 139 running preferably through the member body 124 and/or by connection to the conductive solid core wire 129. The heat pads 134 may be conventional heat pads that utilize electric power, but are preferably less than 1 inch in size, and ideally around ¼ inch, to concentrate the stimulation of temperature change to a specific position.

A number of exposed electrodes 136 may be located at the body tip 126 or along the outer surface of the body 124. The electrodes 136 preferably deliver low frequency electric (DC and/or AC) pulses to a human skin surface. Similar to the vibration motors 132 and heat pads 134, the electrodes 136 are preferably in electric communication with the power source 114, which may be accomplished through wired connections 139 running preferably through the member body 124. The electrodes 136 may be assemblies known in the art.

Where electrically activated stimulation features are utilized, the solid core wire 129, in addition to being plastically deformable and positionable, may also optionally act as a common ground connection for such features. The member body 124 may also include separate electric member body wires 139 to carry power from the power source 114 to the stimulation feature 130. Preferably, each stimulation feature 130 has its own wire(s) 139 connected to the power source 114 and the microprocessor 150 (or the microprocessor 150 controls the amount of power provided). Thus, the user is preferably able to interface with the microprocessor 150 (e.g., through a physical or virtual user interface) to designate which wires 139 receive power and how much, allowing the user to activate certain stimulation features 130 to their desired intensity.

Finally, the stimulation features 130 may optionally or additionally include a number of textured areas 138 for tactile stimulation. Examples of such textures include ridged sections, bumps, and bristles (not shown). A more complex textured area 138 may feature directional fabrics 138a that provide simulation based on how they interact with a user. For example, a number of rounded spikes may protrude perpendicularly from the member body 124 and be uniformly curved in a particular direction. In this way, brushing such an embodiment of a textured area 138 across human skin in a direction opposite to the curve of the spikes may feel smooth to a user. However, brushing this area 138 across human skin in an opposition direction (e.g., a direction that is the same as the direction of the curve of the spikes) will cause increased force concentration between the spike tips and the skin, leading to a rougher, prickly feel. The textured areas 138 may be formed from the same material as the member body 124 and may be manufactured separately and fastened to the body 124 or together with the body 124 as a unitary piece. Additionally or alternatively, textured materials may be fastened to the body 124. Unlike the other stimulation features 130, the textured areas 138 may not rely on electric current and thus may not need to be in electric communication with the power source 114. The textured areas 138 may be placed anywhere on (or formed in) the outer surface of the member body 124.

Textured areas 138 may, however, also provide electrical stimulation, such as by including electrodes 136. Both the textured areas 138 and the electrodes 136 may be made of a different silicone material than the member body 124, preferably with a conductive form of silicone. In this way, the textured areas 138 and electrodes 136 may provide electric impulses for stimulation without the need for any exposed metal. The textured areas 138 and/or electrodes 136 may be formed at the same time as the member body 124 (or in a two-shot process) to provide a water-tight seal between the stimulation features 130 and the member body 124.

In some contemplated embodiments (not shown), the stimulation features 130 may share a common shape, such as a rectangle or square, and may be detachable from the member body 124. The body 124 may also include designated areas in which to place a stimulation feature 130. These areas may be marked on the outer surface of the body 124 or may be sections embedded into the body 124 configured to receive one of the commonly shaped stimulation feature 130. In such embodiments, the user may choose which stimulation feature 130 to place on the member body 124 and where to place them, as they are may be interchanged in the same designated area. The designated areas would also preferably include electrical connections leading to the solid core wire 129 or wires 139, so as to place the chosen stimulation feature 130 in electric communication with the power source 114.

The electric and mechanical connection between the elongated member 120 and the wrist strap 110 may be accomplished through the connector band 140. The connector band 140 is preferably made of the same material as the elongated member 120 (or in any event preferably an electrically insulative material) and may be positioned along the front or back of the user's hand 10. The band 140 may cover any amount of the back of the user's hand 10 (i.e. the entirety of the user's hand 10, only a small portion as a thin strip leading to the one or more elongated members 120, or any amount in between). One or more wires 142 embedded in the band 140 serve to put the elongated members 120 in electric communication with the power source 114 located on the wrist strap 110. In embodiments in which the elongated members 120 contain wires 139, the band wires 142 may continue through the band 140 and into the elongated member 120 to carry electric power to the stimulation features 130. The band 140 also keeps the one or more elongated members 120 in mechanical connection with the strap 110, to assist in maintaining engagement with the finger(s).

Optionally included in the system 100 are a microprocessor 150, which may be in communication with a smart device application (not shown). The microprocessor 150 is preferably one known in the art and may be located proximate the power source 114 on the wrist strap 110. The microprocessor 150 may be used to modulate the flow of power between the power source 114 and the stimulation features 130 that use electric power, providing more or less intense stimulation as desired. Ideally, the microprocessor 150 includes a remote control (not shown) and/or voice activation (not shown) to allow the user or another person to control the power output from the power source 114 and which stimulation feature(s) 130 will receive power. Additionally or alternatively, a smart device (e.g., smartphone) application may be connected to the microprocessor 150 via RF technology (e.g. Bluetooth™) and may be used in a similar fashion to control power output and delegation.

The system 100 may further optionally include lights (e.g. LED lights) (not shown) to provide visual aesthetic stimulation. The lights are preferably in electronic communication with the power source 114 and the microprocessor 150 and may be programmed to change colors when desired. Further, the lights may reflect certain states of the system 100. For example, the lights may glow a certain color when the power source 114 is outputting a higher amount of power, reflecting more intense stimulation, and a different color when the power source 114 is outputting less power, reflecting less intense stimulation. These color options are ideally pre-programmed into the microprocessor 150 and/or programmable by the user through the remote control, voice activation, and/or smart device application.

In use, a stimulation system according to the present invention may fit onto a hand of a user and the one or more stimulation features 130 may be used to stimulate the body of the user or another. The system 100 is especially advantageous in the amount of body surface area it may affect at any one time. The user begins by inserting a fingers into each sheath 122 of the elongated member(s) 120 provided. The user may then activate the power source 114 (e.g. put batteries into the mount 118 and/or activate a power switch) and may selectively activate stimulation features 130 and related intensity levels using a physical user interface (e.g., buttons/switches/capacitive display supported by the strap 110 or connection band 140), remote control, voice activation, and/or smart device application. The user may then choose a desired area of a human body (from as small as less than ¼″, such as if a single member 120 (with rounded or pointed tip) is used, to multiple points on the body that may be separated by up to, for example, twenty-four inches) to stimulate and bend the elongated members 120 in such an arrangement as may be suitable for their needs. The user may change the positioning of the elongated members 120 or the settings of the stimulation features 130 as many times as they desire in any given session. After use, the user may power off the power source 114 and remove their fingers from the sheaths 122.

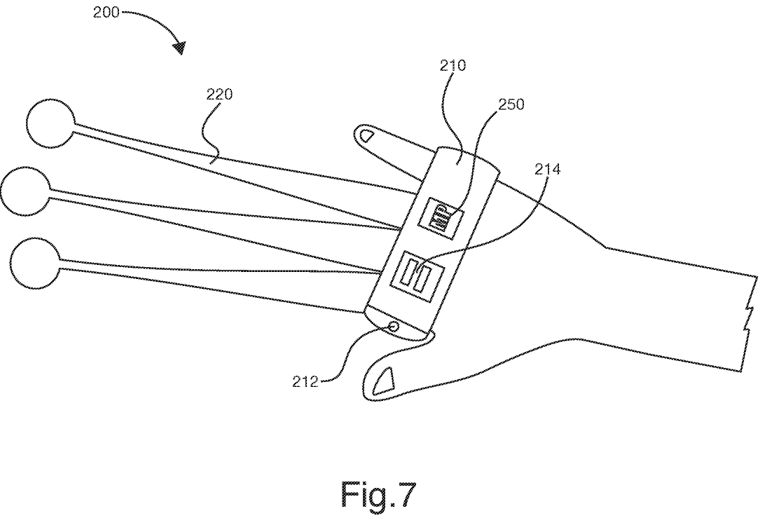

A second embodiment of a body stimulation system is shown in FIG. 7. The body stimulation system 200 comprises many elements at least similar (if not identical) to those provided in the first embodiment including a strap 210, one or more elongated members 220, and optional microprocessor 250, smart device application, and lights. However, the strap 210 of this system 200 is configured to fit around a user's hand 10 proximate to the knuckles at the base of the fingers 30. This configuration allows the one or more elongated members 220 to be fastened directly to the hand strap 210, preferably eliminating the need for the connector band 140. The power source 214 is still in electronic communication with the elongated member 220, just without the need for wires 142 running along the back of the user's hand. Apart from the location of the strap 210, all other aspects of the system 200 work at least substantially as described in connection with the first embodiment.

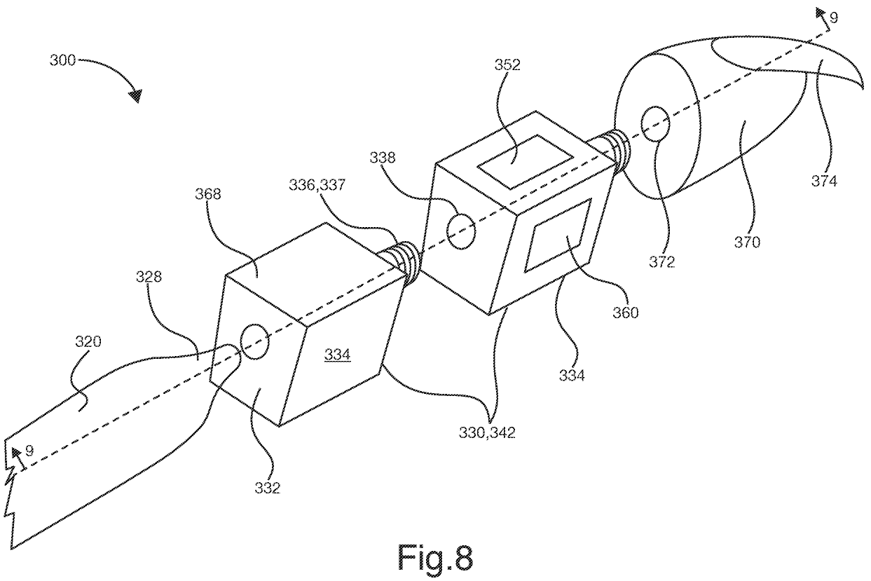

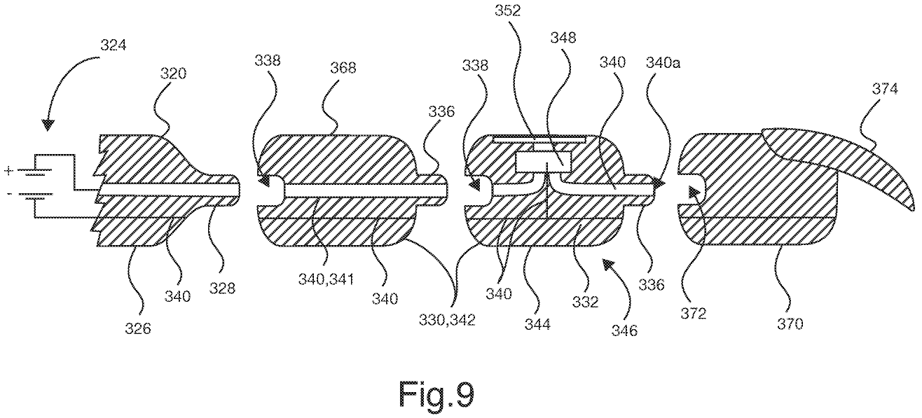

A third embodiment of a body stimulation system according to the present invention is shown in FIGS. 8 and 9. The body stimulation system 300 comprises a finger sheath 320, one or more extensions 330, and a terminal extension 370. The finger sheath 320 is preferably made of the same material as the moldable body 124 described above. The finger sheath 320 may be self-supported on a user's finger, wherein no other supporting structures hold the sheath 320 onto the user's finger. Such a sheath 320 may feature a smooth or textured inside surface 322 (not shown), which frictionally engages the user's finger (with an operatively sufficient friction force) to prevent the sheath 320 from falling off during use. This sheath 320 may also feature a power source (not shown), such as one or more button batteries. Alternatively, the sheath 320 may be supported by, and in electrical communication with, a strap 110/210 as in embodiments one or two described above. The sheath 320 further preferably comprises embedded or exposed electrical connections 326, connected to the strap 110/210 and/or power source 324, running to a connecting member 328. As will be understood, the electrical connections 326 are (preferably at least two in number) operatively sufficient to provide power and ground connections to or through successively connected extensions 330.

The extensions 330 each have a substantially similar cross-section, such as block or ovoid-shaped units of the same material as the finger sheath 320. The extensions 330 may be described in two categories based on their function: active extensions 344 and passive extensions 368. However, both categories of extensions 330 share some similar features. For example, each extension 330 comprises a body 332 with an outer surface 334. Extensions 330 are preferably coupled at least mechanically, and optionally, electrically, to at least one other extension 330 or the sheath 320. Protruding from one end of the body 332 is a first coupling mechanism, such as a peg 336. On the opposite end of the body 332 is a second coupling mechanism, mateable with the first, such as an inlet 338 which is configured to selectively receive and retain the peg 336. The peg 336 may comprise a retention member 337, which adds resistance between the peg 336 and the inlet 338 and/or prevents the two extensions 330 from disconnecting. The retention member 337 may be provided as an active (e.g., spring-biased) or passive detent (partially or completely annular) that adds resistance within the inlet 338 or cooperates with an indent therein, threads wherein the peg 336 mates with threads provided in the inlet 338, a radially activatable slide-lock mechanism between the peg 336 (e.g., as a T-stud) and the inlet 338 (e.g., as a cooperating C-groove), or some combination of the like. What is to be understood is that the extensions 330 are preferably mechanically and electrically coupled to the sheath 320 and/or one or more additional extensions 330 and/or the terminal extension 370.

While each body 332 is preferably a substantially solid (e.g., molded) unit of material, electrical connections 340 preferably run through the body 332 from the peg 336 to the inlet 338, and provide power to any features in the extension 330 requiring same. The extensions 330 may also feature a solid core wire 341 similar to the embodiments above, to allow the extensions 330 to bend slightly and hold their shape. In use, the inlet 338 may receive the peg 336 of another extension 330 or the connecting member 328, wherein the electrical connections 340 of the finger sheath 320 and extension 330 (or extension 330 and another extension 330) are in electrical communication through an electronic connector 340a. This connector 340a may be in the form of a plug or contact connector. In this way, it is possible to build a chain of extensions 342 from the finger sheath 320, wherein each extension 330 is in electrical communication with the power source 324.

Active extensions 344 are characterized in that they are capable of performing some action, in addition to mechanically extending the device. For example, one active extension 344 may be a vibrating extension 346, wherein a small vibratory motor 348 is embedded in the extension body 332 and coupled to the electrical connections 340. Another active capability may be warming or heating, wherein a heating pad 352 is embedded in (or just below) the outer surface 334 of the extension body 332, again powered by the electrical connections 340. Other contemplated active extensions may include active and return electrodes exposed on the outer surface to provide continuous or pulsed electrical current between them when a conductive surface (e.g., sweaty skin) is disposed therebetween.

Finally, some active extensions 344 may additionally or alternatively feature a single or multi-color LED, visible through the outer surface 334. An active extension 344 may also feature more than one active component at one time, such as a vibrating extension 346 that also features a heating element 352.

Passive extensions 368, on the other hand, are characterized in that they do not feature an active functionality. Instead, the passive extensions 368 comprise just the body 332 and embedded electrical extensions 340, and may further include textured extensions 358, featuring directional fabrics 360 as described in previous embodiments. Passive extensions 368 may be used to simply lengthen the chain of extensions 342 and to strategically arrange active extensions 344 on specific areas along the chain 342 as desired by the user.

Finally, the chain of extensions 342 may end with the terminal extension 370. The terminal extension 370 preferably comprises an inlet 372, similar to the extensions 330, but does not include any further coupling mechanism. Rather, the terminal extension 370 includes a free end opposite the inlet 372. Further, the terminal extension 370 is preferably a solid unit of the same material as the finger sheath 320, which may or may not include electrical connections embedded. In this way, the terminal extension 370 may serve to insulate the electrical connections 340 of the extensions 330 to prevent unintentional contact with a user. The rounded end of the end cap 370 may further comprise passive stimulants 374, such as a hard plastic nail that is slightly sharpened or dulled. These stimulants 374 act to provide further types of stimulation from the system 300. Static stimulants 374 may even be retractable into the terminal extension 370. An alternative type of stimulant 374 may be an ice cube frozen to the terminal extension 370, about the sharpened nail or instead of the nail, so that the free end of the terminal extension 370 is at least temporarily formed from ice during use.

A kit (e.g., sealed or selectively placed in the same compartmented or uncompartmented package) comprising one or more of the straps 110/210, elongated members 120/220, connector band 140, finger sheath 320, extension(s) 330, and terminal extension 370, and/or an ice tray mold to be used to form an ice cube to be supported by an extension. For instance, an extension 330/370 may be received by an ice cube tray filled at least partially with water to mold an ice formation (cube or otherwise) onto the extension.

The foregoing is considered as illustrative only of the principles of the invention. Furthermore, because numerous modifications and changes will readily occur to those skilled in the art, it is not desired to limit the invention to the exact construction and operation shown and described. While the preferred embodiment has been described, the details may be changed without departing from the invention.

I claim:

1. A system for manually supported stimulation comprising:
at least one elongated member comprising:
a finger sheath configured to receive a human finger and be at least partially supported thereby,
a body extending from the finger sheath to a free end, the finger sheath and body extending generally coaxially along a longitudinal length, the longitudinal length being at least three inches, and
a first stimulation feature supported by the body.

2. The system of claim 1, wherein the longitudinal length is three inches to twelve inches.

3. The system of claim 2, wherein the longitudinal length is five inches to twelve inches.

4. The system of claim 1, wherein the first stimulation feature is located closer to the free end than to the finger sheath.

5. The system of claim 1, further comprising a power supply electrically coupled to the first stimulation feature.

6. The system of claim 5, further comprising a strap, the power supply being supported by the strap.

7. The system of claim 6, wherein the strap is configured to be worn on one or more of a human wrist and a human hand.

8. The system of claim 6, further comprising:
a connector band in mechanical and electronic communication with the strap and the elongated member.

9. The system of claim 5, wherein the power supply comprises a rechargeable battery.

10. The system of claim 5, wherein the first stimulation feature and the power supply are electrically coupled by a wire that extends along a majority of the longitudinal length, the wire further capable of supporting the body in a selective plastically deformed position.

11. The system of claim 1, wherein the first stimulation feature comprises at least one of a vibration motor, a heat pad, an electrically conductive surface, and a textured area.

12. The system of claim 1, wherein the elongated member comprises a second stimulation feature.

13. The system of claim 12, wherein the second stimulation feature comprises at least one of a vibration motor, a heat pad, an electrically conductive surface, and a textured area.

14. The system of claim 12, wherein the second stimulation feature is different than the first stimulation feature.

15. A system for manually supported stimulation comprising:
a finger sheath configured to receive a human finger and be at least partially supported thereby,
a body extending from the finger sheath to a free end, the finger sheath and body extending generally curvilinearly coaxially along a longitudinal length, the longitudinal length being at least three inches, and
a first stimulation feature supported by the body,
wherein the body comprises:
a first extension member coupled to the finger sheath; and
a second extension member coupled to the first extension member and forming the free end.

16. The system of claim 15, further comprising one or more additional extension members disposed between the first extension member and the second extension member.

17. The system of claim 16, wherein the first and second extension members are mechanically coupled.

18. The system of claim 17, wherein the first and second extension members are electrically coupled.

19. The system of claim 16, wherein at least one of the additional extension members is electrically coupled to the first extension member.

20. The system of claim 15, wherein the first stimulation feature is an active stimulation feature.

21. The system of claim 20, wherein the first stimulation feature is at least one of a vibrating motor, a heating pad, an electrically conductive surface, and a light.

22. The system of claim 15, wherein the first stimulation feature is a passive stimulation feature.

23. The system of claim 22, wherein the first stimulation feature is at least one of a textured area and an ice cube.
